(19) United States
(12) Patent Application Publication
(10) Pub. No.: US 2022/0192536 A1
(43) Pub. Date: Jun. 23, 2022

(54) SYSTEM FOR MONITORING RESPIRATORY CONDITIONS AND METHOD THEREOF

(71) Applicant: RESMETRIX MEDICAL LTD., Haifa (IL)

(72) Inventors: Zvi REZNIC, Tel-Aviv (IL); Carmit LEVY, Kiryat Ono (IL)

(21) Appl. No.: 17/612,472

(22) PCT Filed: Jun. 17, 2020
(86) PCT No.: PCT/IL2020/050668

§ 371 Date: Nov. 18, 2021

Related U.S. Application Data
(60) Provisional application No. 62/862,171, filed on Jun. 17, 2019.

(51) Int. Cl. A61B 5/08; A61B 5/113; A61B 5/00

(52) U.S. Cl. CPC A61B 5/0809; A61B 5/1135; A61B 5/7282; A61B 5/6831; A61B 5/6823

(57) ABSTRACT

There is provided a method and system of monitoring a respiratory condition of a subject, the method including obtaining input data indicative of an alternating signal representative of change of length of at least part of a contour along a subject's chest wall and/or abdominal wall during one or more breathing cycles, the input data generated by periodically measuring the change of length during the breathing cycles; analyzing the input data to detect an irregular breathing pattern, the irregular breathing pattern represented by at least one local change in the form of the alternating signal during an expiratory phase of at least one of the one or more breathing cycles; and determining a respiratory condition of the subject based on a result of the detection.

### TECHNICAL FIELD

The presently disclosed subject matter relates, in general, to the field of respiratory monitoring, and more specifically, to methods and systems for respiratory monitoring based on data analysis.

### BACKGROUND

Respiratory monitoring is critical for patients, especially those with severe medical conditions which, in turn, affect the lungs and overall pulmonary functions. Respiratory data can provide valuable information concerning the progression of a disease or injury affecting a patient, which is very useful for assessing, diagnosing and treating respiratory symptoms driven by such disease or injury. For instance, it is proven that post-operative respiratory volume monitoring can help predict the risk of life-threatening complications in post-surgical patients, which may occur hours after the patients are considered stabilized after surgery. Urgent medical resuscitation is essential in such cases to prevent respiratory failure and death.

Nevertheless, respiratory monitoring could also be important for individuals to use during normal daily routines so as to be able to capture respiratory events which may be indicative of a patient's state of respiratory health and reveal adverse conditions which might otherwise go unnoticed. For instance, the breathing of babies at risk for Sudden Infant Death Syndrome (SIDS) is difficult to monitor, and accurate non-invasive monitoring at home may be life-saving. Obstructive sleep apnea in adults and children requires accurate respiratory monitoring in order to diagnose and treat. Again, accurate non-invasive respiratory monitoring can facilitate this.

Existing monitoring systems and methods are normally complex, inconvenient, and costly to be effectively and widely used by patients or individuals. In addition, existing respiratory assessment in such systems is normally based on known respiratory metrics, and in some cases can be computationally cumbersome and inaccurate.

### GENERAL DESCRIPTION

In accordance with certain aspects of the presently disclosed subject matter, there is provided a computerized method of monitoring a respiratory condition of a subject, the method performed by a processor and memory circuitry (PMC) and comprising: obtaining input data indicative of an alternating signal representative of change of length of at least part of a contour along a subject's chest wall and/or abdominal wall during one or more breathing cycles, the input data generated by periodically measuring the change of length during the breathing cycles; analyzing the input data to detect an irregular breathing pattern, the irregular breathing pattern represented by at least one local change in the form of the alternating signal during an expiratory phase of at least one of the one or more breathing cycles; and determining a respiratory condition of the subject based on a result of the detection.

In addition to the above features, the method according to this aspect of the presently disclosed subject matter can comprise one or more of features (i) to (xvi) listed below, in any desired combination or permutation which is technically possible:
(i). The change of length is measured using an electrical signal, and at least part of the signal path of the electrical signal contours at least part of the chest wall and/or abdominal wall.
(ii). The electrical signal flows through a band attachable to the chest wall and/or abdominal wall of the subject.
(iii). The input data is generated by, for at least one time interval during a breathing cycle, measuring an attribute of the electrical signal received at the beginning and the end of the time interval, and calculating a change in length of the signal path during the time interval based on the measurements of the attribute received at the beginning and the end of the time interval.
(iv). The at least one local change comprises one or more of the following: local bend, local peak, and change of slope.
(v). The analyzing comprises calculating first derivative data indicative of change rate of the length of at least part of a contour with respect to time, and detecting the at least one local change based on the first derivative data.
(vi). The at least one local change is characterized by second derivative data thereof passing a predefined threshold, the second derivative data being indicative of a change rate of first derivative data with respect to time, the first derivative data indicative of change rate of the length of at least part of a contour with respect to time.
(vii). The at least one local change comprises a positive local peak, and the analyzing comprises detecting the positive local peak by calculating topographic prominence of positive peaks in the one or more breathing cycles, generating a distribution of the topographic prominence of the positive peaks, and identifying the positive local peak based on the distribution.
(viii). The at least one local change comprises a negative local peak, and the analyzing further comprises detecting the negative local peak by modifying the alternating signal by multiplying the alternating signal with a negative number so as to transform negative peaks in the one or more breathing cycles into positive peaks, and performing the calculating, generating and identifying on the modified alternating signal to detect the negative local peak.
(ix). The at least one local change comprises a positive local peak, and the analyzing comprises detecting the positive local peak by searching for two consecutive positive peaks using first derivative data, wherein a time difference between the two consecutive positive peaks is less than a predetermined threshold.
(x). The at least one local change comprises a local bend, and the analyzing comprises detecting the local bend by modifying the alternating signal by adding a linear function of time thereto so as to transform the local bend into a local peak, and detecting the transformed local peak on the modified alternating signal.
(xi). The at least one local change comprises one or more local bends, and the analyzing comprises iteratively modifying the alternating signal by adding respective linear functions of time, giving rise to respective modified alternating signals, detecting the one or more local bends based on a modified alternating signal selected from the respective modified alternating signals which maximizes the number of positive peaks in the one or more breathing cycles in the selected modified alternating signal.
(xii). The at least one local change comprises a local change of slope, and the analyzing comprises detecting the local change of slope using first derivative data and/or second derivative data.
(xiii). The method further comprises counting the number of occurrences of the irregular breathing pattern during the one or more breathing cycles, and determining the respiratory condition based on the counted number.
(xiv). The method further comprises analyzing the input data to detect an additional irregular breathing pattern represented by irregular change of said length of at least part of a contour between the beginning and end of the expiratory phase.
(xv). The respiratory condition is selected from a list of pre-defined set of conditions indicative of different types of deterioration of lung function.
(xvi). The method further comprises generating an alert upon determination of a pre-defined respiratory condition, and sending the alert to an external device.

In accordance with other aspects of the presently disclosed subject matter, there is provided a computerized system of monitoring a respiratory condition of a subject, the system comprising a processor and memory circuitry (PMC) configured to: obtain input data indicative of an alternating signal representative of change of length of at least part of a contour along a subject's chest wall and/or abdominal wall during one or more breathing cycles, the input data generated by periodically measuring the change of length during the breathing cycles; analyze the input data to detect an irregular breathing pattern, the irregular breathing pattern represented by at least one local change in the form of the alternating signal during an expiratory phase of at least one of the one or more breathing cycles; and determine a respiratory condition of the subject based on a result of the detection.

This aspect of the disclosed subject matter can comprise one or more of features (i) to (xvi) listed above with respect to the method, mutatis mutandis, in any desired combination or permutation which is technically possible.

In accordance with other aspects of the presently disclosed subject matter, there is provided a non-transitory computer readable storage medium tangibly embodying a program of instructions that, when executed by a computer, cause the computer to perform a method of monitoring a respiratory condition of a subject, the method comprising: obtaining input data indicative of an alternating signal representative of change of length of at least part of a contour along a subject's chest wall and/or abdominal wall during one or more breathing cycles, the input data generated by periodically measuring the change of length during the breathing cycles; analyzing the input data to detect an irregular breathing pattern, the irregular breathing pattern represented by at least one local change in the form of the alternating signal during an expiratory phase of at least one of the one or more breathing cycles; and determining a respiratory condition of the subject based on a result of the detection.

This aspect of the disclosed subject matter can comprise one or more of features (i) to (xvi) listed above with respect to the method, mutatis mutandis, in any desired combination or permutation which is technically possible.

In accordance with other aspects of the presently disclosed subject matter, there is provided a device capable of monitoring a respiratory condition of a subject, the device attachable to the subject's chest wall and/or abdominal wall and comprising a transmitter, a receiver, and a processor and memory circuitry (PMC) operatively connected thereto, wherein: the transmitter is configured to transmit an electronic signal to the receiver during one or more breathing cycles; the receiver is configured to receive the electronic signal transmitted from the transmitter; and the PMC is configured to: periodically measure change of length of at least part of a contour along the subject's chest wall and/or abdominal wall during the one or more breathing cycles based on the received electronic signal, and generate data indicative of an alternating signal representative of the change of length; analyze the data to detect an irregular breathing pattern, the irregular breathing pattern represented by at least one local change in the form of the alternating signal during an expiratory phase of at least one of the one or more breathing cycles; and determine a respiratory condition of the subject based on a result of the detection.

This aspect of the disclosed subject matter can comprise one or more of features (i) to (xvi) listed above with respect to the method, mutatis mutandis, in any desired combination or permutation which is technically possible.

### BRIEF DESCRIPTION OF THE DRAWINGS

In order to understand the present disclosure and to see how it may be carried out in practice, embodiments will now be described, by way of non-limiting example only, with reference to the accompanying drawings, in which:

FIG. 1 schematically illustrates a functional block diagram of a respiratory monitoring system in accordance with certain embodiments of the presently disclosed subject matter.

FIG. 2 illustrates a generalized flowchart of monitoring a respiratory condition in accordance with certain embodiments of the presently disclosed subject matter.

FIG. 3 illustrates a generalized flowchart of detecting a positive local peak in accordance with certain embodiments of the presently disclosed subject matter.

FIG. 4 illustrates a generalized flowchart of detecting a local bend in accordance with certain embodiments of the presently disclosed subject matter.

FIGS. 5A and 5B illustrate an exemplified measurement device attached to an individual's chest wall in accordance with certain embodiments of the presently disclosed subject matter.

FIGS. 6A, 6B and 6C illustrate exemplified measurement devices attached to an individual's chest wall in accordance with certain embodiments of the presently disclosed subject matter.

FIGS. 7A and 7B illustrate examples of input data in the form of an alternating signal in accordance with certain embodiments of the presently disclosed subject matter.

FIG. 8 illustrate an example of a local peak in the form of the alternating signal during an expiratory phase of a breathing cycle in accordance with certain embodiments of the presently disclosed subject matter.

FIG. 9 illustrates detection of local peaks using first derivative data and/or second derivative data in accordance with certain embodiments of the presently disclosed subject matter.

FIG. 10 illustrates an example of a local bend in the form of the alternating signal during an expiratory phase of a breathing cycle in accordance with certain embodiments of the presently disclosed subject matter.

FIG. 11 illustrates an example of a modified alternating signal in accordance with certain embodiments of the presently disclosed subject matter.

FIG. 12 illustrates detection of a local bend using first derivative data and/or second derivative data in accordance with certain embodiments of the presently disclosed subject matter.

FIG. 13 illustrates an example of local change of slope in the form of the alternating signal during an expiratory phase of a breathing cycle in accordance with certain embodiments of the presently disclosed subject matter.

FIG. 14 illustrates detection of a local change of slope using first derivative data and/or second derivative data in accordance with certain embodiments of the presently disclosed subject matter.

FIG. 15 illustrates an example of measuring the prominence of a peak in accordance with certain embodiments of the presently disclosed subject matter.

### DETAILED DESCRIPTION OF EMBODIMENTS

In the following detailed description, numerous specific details are set forth in order to provide a thorough understanding of the present disclosure. However, it will be understood by those skilled in the art that the presently disclosed subject matter may be practiced without these specific details. In other instances, well-known methods, procedures, components and circuits have not been described in detail so as not to obscure the presently disclosed subject matter.

Unless specifically stated otherwise, as apparent from the following discussions, it is appreciated that throughout the specification discussions utilizing terms such as “obtaining”, “analyzing”, “determining”, “monitoring”, “measuring”, “calculating”, “detecting”, “identifying”, “modifying”, “multiplying”, “performing”, “searching”, “adding”, “counting”, “sending”, or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities including, by way of non-limiting example, the respiratory monitoring system and parts thereof disclosed in the present application.

The operations in accordance with the teachings herein can be performed by a computer specially constructed for the desired purposes or by a general purpose computer specially configured for the desired purpose by a computer program stored in a non-transitory computer readable storage medium.

The terms “non-transitory memory”, “non-transitory storage medium” and “non-transitory computer readable storage medium” used herein should be expansively construed to cover any volatile or non-volatile computer memory suitable to the presently disclosed subject matter.

Embodiments of the presently disclosed subject matter are not described with reference to any particular programming language. It will be appreciated that a variety of programming languages may be used to implement the teachings of the presently disclosed subject matter as described herein.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are described in the context of separate embodiments, can also be provided in combination in a single embodiment. Conversely, various features of the presently disclosed subject matter, which are described in the context of a single embodiment, can also be provided separately or in any suitable sub-combination. In the following detailed description, numerous specific details are set forth in order to provide a thorough understanding of the methods and apparatus.

In embodiments of the presently disclosed subject matter, one or more stages illustrated in the figures may be executed in a different order and/or one or more groups of stages may be executed simultaneously, and vice versa.

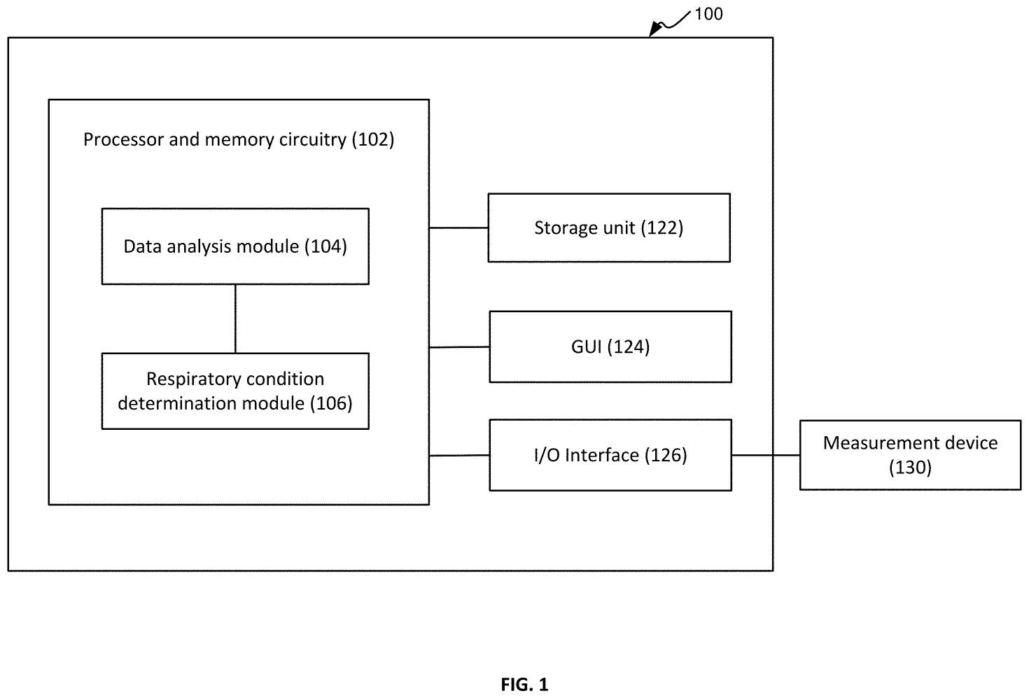

Bearing this in mind, attention is drawn to FIG. 1, schematically illustrating a functional block diagram of a respiratory monitoring system in accordance with certain embodiments of the presently disclosed subject matter.

The system 100 illustrated in FIG. 1 is a computer-based system for automatically monitoring a respiratory condition of a subject. System 100 can receive certain measurement data during the subject's breathing cycle(s), and determine a respiratory condition thereof based on analysis of the data. The term subject used herein can refer to any living subject that can perform respiration actions, such as, e.g., a living human being or a living animal. When a subject breathes, the chest expands and contracts causing an increase or decrease respectively in the length of the contour of the chest wall (e.g., the circumference of the chest), but also the diaphragm muscle which is located between the thorax and the abdomen expands and contracts causing an increase or decrease respectively in in the length of the contour of the abdominal wall (e.g., the circumference of the abdomen). Therefore, respiration can be done as a combination of movement of the thoracic cavity and of the diaphragm. According to certain embodiments, the system 100 can be configured to obtain, from a device or apparatus 130, input data indicative of an alternating signal representative of change of length of at least part of a contour along the subject's chest wall and/or abdominal wall during one or more breathing cycles of the subject.

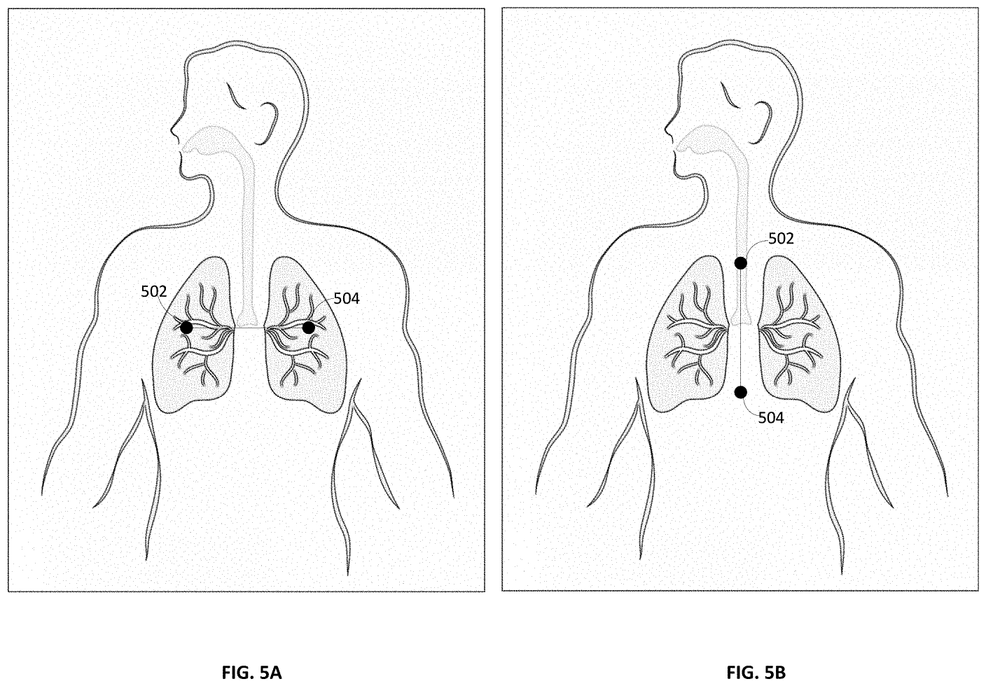

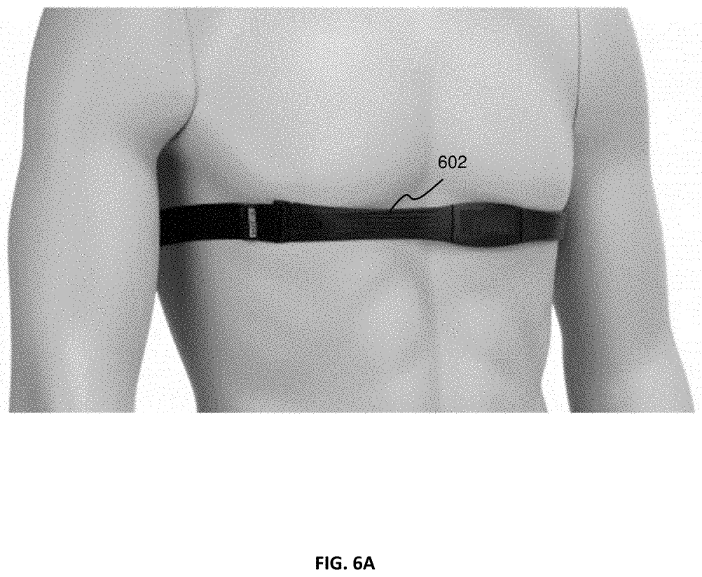

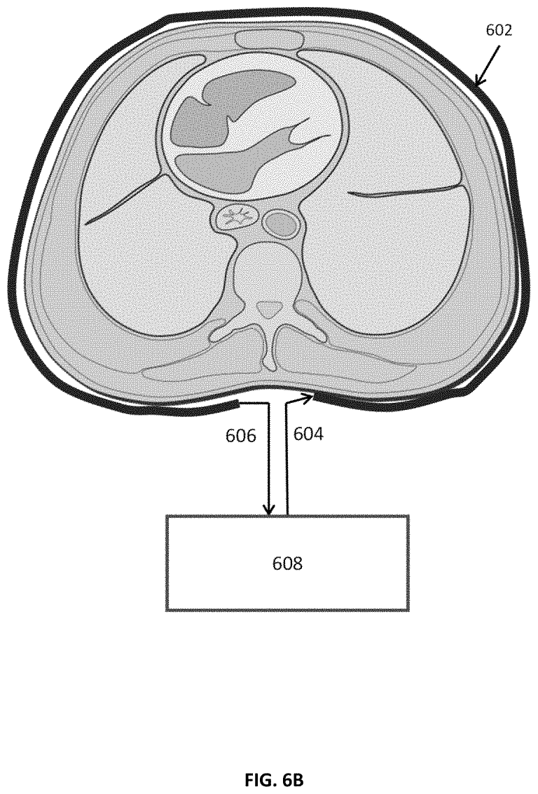

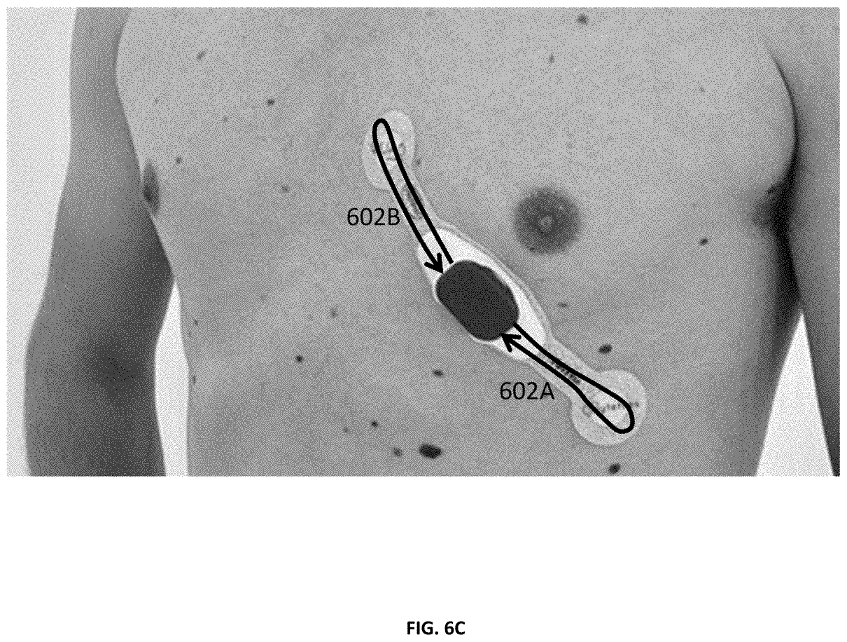

Device 130 is configured to periodically measure the change of length of at least part of a contour along the subject's chest wall and/or abdominal wall during the breathing cycles. In some cases, the device 130 can be attached to the subject's body for performing such measurements. For instance, the device can be worn by the subject, or alternatively it can be stuck or glued to the body, as exemplified in FIGS. 5, 6A, 6B and 6C. Therefore, device 130 is also referred to herein as a measurement device, an attachable device, or a wearable device.

In some embodiments, device 130 can be configured to measure the change of length using an electrical signal, such as, e.g., a radio frequency (RF) signal. At least part of signal path of the electrical signal contours at least part of the chest wall and/or abdominal wall. For simplicity's sake, certain embodiments of the subject matter are described herein with reference to an RF signal where the range of frequencies can be between 3 Khz to 300 Ghz, but any electronic signal can be used additionally or alternatively, mutatis mutandis.

According to certain embodiments, the device 130 can comprise at least one pair of transmitter and receiver. The transmitter and/or the receiver can be attached externally to a portion of the chest wall and/or the abdominal wall, and/or anywhere else which enables measurement of a change of length of at least part of a contour along the subject's chest wall and/or abdominal wall. During the breathing cycles, the chest wall and/or abdominal wall moves as the volume of the lung changes. The transmitter can be configured to transmit the RF signal. The receiver can be configured to receive the transmitted RF signal from the transmitter. At least part of the path of the signal contours at least part of the chest wall and/or the abdominal wall. This means that at least part of the signal path is on or beside at least part of the chest wall and/or the abdominal wall so that the at least part of the signal path adjusts when the at least part of the chest wall and/or the abdominal wall moves.

For simplicity's sake, certain embodiments of the presently disclosed subject matter are described and illustrated with reference to a subject of a human patient, but in some cases the subject can alternatively be an animal, as aforementioned. Likewise, certain embodiments of the presently disclosed subject matter are described with reference to placement and measurement of device 130 along the chest wall of the subject. However, this is for exemplary purposes only and should not be regarded as limiting the disclosure in any way. In some cases, the placement and measurement can be along the abdominal wall, while in some other cases, the placement and measurement can be along both the chest wall and the abdominal wall. For example, referring to the example above of a transmitter and a receiver, the transmitter may be attached to the surface of the chest wall, while the receiver is attached to the surface of the abdominal wall, such that the signal path starts at the chest wall and ends at the abdominal wall.

Referring to FIGS. 5A and 5B now, there is illustrated an exemplified measurement device attached to an individual's chest wall in accordance with certain embodiments of the presently disclosed subject matter.

In accordance with certain embodiments, the signal is transmitted wirelessly between antennas, and therefore can travel through the air between antennas. A signal that travels through the air between antennas can be considered to be a non-guided signal. However, even when the signal travels through the air between antennas, optionally part of the signal path may not be through the air, e.g. if the signal travels from the transmitter to its associated antenna that is located apart, and/or travels from an antenna associated with the receiver to the receiver that is located apart.

According to certain embodiments, the transmitter 502 (or at least the antenna thereof) and the receiver 504 (or at least the antenna thereof) can be placed on the chest wall of the lung. By way of example, the transmitter 502 and the receiver 504 can be positioned horizontally apart in parallel with the ribs, as shown in FIG. 5A. By way of another example, the transmitter 502 and the receiver 504 can be positioned vertically apart along the direction of the airway, as shown in FIG. 5B. It is to be noted that the placements in FIGS. 5A and 5B are illustrated for exemplary purposes only, and should not be construed to limit the scope of the present disclosure in any way. Accordingly, the transmitter 502 and receiver 504 (or at least respective antennas) can be positioned in any other suitable positions, such as each under an armpit or on the side of the chest, or, alternatively, one attached on the front of the chest, and the other attached on the back of the chest, or in any other relative positions, such as, e.g., in diagonal positions one to the other. In cases where there are multiple pairs of transmitter and receiver, the pairs can be positioned in any suitable places, such as one or more pairs of transmitter 502 and receiver 504 positioned horizontally apart and/or one or more pairs of transmitter 502 and receiver 504 positioned vertically apart, in any suitable positions. Similarly, other compositions of transmitter(s) and receiver(s) can be positioned in any suitable positions.

In accordance with certain embodiments, the transmitter 502 and the receiver 504 can be placed such that there is a line of sight between the antennas. As the signal is not guided, once it leaves the antenna associated with the transmitter 502, the transmitted signal can go in multiple directions, with one of the directions being the line of sight direction, such that at least part of the signal path contours at least part of the chest wall and/or abdominal wall. Possibly there may also be reflected signal(s), due to reflection(s) of the transmitted signal, whose signal path(s) did not contour any part of the chest wall, but which can also be received by the receiving antenna. The signal received directly (line of sight) is significantly larger than the reflected signal(s). Therefore, the line of sight signal is dominant in the received signal, and a measurement of an attribute of the received signal (even if including reflected signal(s)), may be considered to be substantially equivalent to a measurement of an attribute of the direct (line of sight) signal. Moreover, since the line of sight signal is dominant, at least part of the path of the signal that is received can be said to contour at least part of the chest wall, because at least part of the path of the line of sight signal contours at least part of the chest wall, regardless of whether or not the signal that is received included reflected signal(s) whose path(s) do not contour any part of the chest wall.

In accordance with certain embodiments, the transmitter 502 (or at least the antenna thereof) and the receiver 504 (or at least the antenna thereof) can be attached to the chest wall and/or abdominal wall with standard adhesives (e.g., adhesive stickers or patches) or to a stretchable garment worn on the torso.

Refer now to FIGS. 6A and 6B which illustrate another exemplified measurement device attached to an individual's chest wall in accordance with certain embodiments of the presently disclosed subject matter.

According to certain embodiments, the electrical signal used in the present disclosure may be guided. For example, the signal can flow for at least part of the path between the transmitter and the receiver through a belt, or a strap or a band, or similar, attached to a chest wall, so that the path of the signal, through at least part of the strap, contours at least part of the chest wall. The terms belt, strap, band and similar should be construed as equivalent terms for an item wrapped around at least part of the chest and/or abdomen and through which a signal can travel.

FIG. 6A illustrates a strap 602 through which a signal can flow, in accordance with certain embodiments of the presently disclosed subject matter. The strap 602 encircles the chest wall and there is a small central unit (e.g. plastic case) near the chest (e.g. attached to the chest) which includes the transmitter and the receiver. The transmitter generates an electronic signal which is transmitted through the strap 602 to the receiver.

FIG. 6B illustrates an exemplified implementation of a strap through which a signal can flow, in accordance with certain embodiments of the presently disclosed subject matter. As shown, the strap 602 encircles the chest wall. The central unit 608 includes the transmitter and the receiver (not illustrated separately). The transmitter in the central unit 608 generates an electronic signal 604 which is transmitted through the strap 602 to the receiver. The signal 606 exiting the strap 602 enters into the receiver which is inside the central unit 608. The receiver can determine measurements for one or more attribute(s) of the received signal 606. Optionally, attribute(s) of the received signal 606 can also be compared to attribute(s) of the transmitted signal 604.

It is to be noted that although the strap 602 is illustrated as encircling the entire chest wall, in certain embodiments, there may not be a total encirclement of the chest wall by a strap. For instance, a strap can be attached to less than 100% (e.g. 1.5%, 5%, 50%, 70%, etc.) of the chest wall. In another instance, there can be more than one chest strap (e.g. one attached to the front of the chest wall and one to the back of the chest wall, e.g. one attached horizontally, and one attached vertically, etc.). In embodiments with a plurality of straps, a separate pair of transmitter and receiver can be located on the two ends of any given strap, two or more straps can share transmitter(s) and/or receiver(s), or one or more straps can have separate transmitter(s) and/or receiver(s), while two or more other straps can share transmitter(s) and/or receiver(s).

It is to be noted that in embodiments with a strap, the signal received by the receiver is typically, although not necessarily, made up solely of the signal that traveled through the strap. In other words, the signal received by the receiver typically, although not necessarily, does not include any reflected signals whose path(s) did not contour any part of the chest wall, as may be the case when the signal is non-guided due to having been wirelessly transmitted. As mentioned above, the path of the signal through at least part of the strap contours at least part of the chest wall, and therefore at least part of the path of the signal that is received contours at least part of the chest wall and/or abdominal wall.

It should be understood that when any of transmitter, receiver, antenna, adhesive, central unit and/or strap is described as “attached”, “positioned”, “placed” or similar, to the chest or chest wall, under the armpit, on a circumference of a chest wall, etc., the subject matter does not limit the manner of placement as long as the placement enables the signal path length to change when the chest moves during a breathing cycle. For example, any of the above may touch the skin of the patient, may touch a garment worn by the patient, may be integrated in a garment worn by the patient; may be integrated in an adhesive, strap, or central unit that touches the skin, touches a garment worn by the patient, or is integrated in a garment worn by the patient, etc. In another example, the placement may cause the signal to travel in a horizontal direction, in a vertical direction, diagonally, unevenly, up and down, and/or slanted, etc.

According to certain embodiments, multiple pairs of transmitter and receiver (and/or one or more sets of transmitter and corresponding multiple receivers and/or one or more sets of receiver and corresponding multiple transmitters) can be applied to make the measurement and calculation of change of length more accurate.

It is to be appreciated that the present disclosure is not limited by the specific number, type, structure of the measurement device, nor by the specific generation methods of the input data by the measurement device.

FIG. 6C illustrates an exemplified measurement device implemented as a patch attached to the chest wall using adhesive material in accordance with certain embodiments of the presently disclosed subject matter. There is shown a small central unit (e.g. plastic case) near the center of the patch which includes two transmitters and two receivers. There are two straps 602A and 602B, and in each strap, the signal flows in a certain loop, that starts and ends at the central unit. The device can then monitor, individually, changes in the lengths of strap 602A and changes in the length of strap 602B. In this example, the signal path in each strap goes in one direction, away from the central unit, and then it turns, and starts flowing back, towards the central unit. One advantage of having two pairs of transmitter and receiver is the ability to have two separate contours covering different ranges, thereby being able to collect more measurement information to be provided to the system 100.

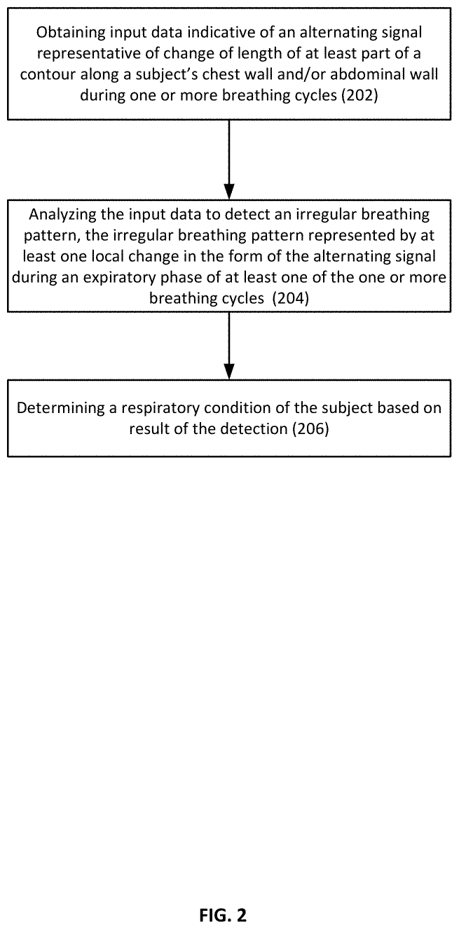

Continuing with the description of FIG. 1, system 100 can comprise a processor and memory circuitry (PMC) 102 operatively connected to a hardware-based I/O interface 126 and a storage unit 122. The PMC 102 is configured to provide processing necessary for operating system 100 which is further detailed with reference to FIGS. 2-4. PMC 102 comprises a processor (not shown separately) and a memory (not shown separately). The processor of PMC 102 can be configured to execute several functional modules in accordance with computer-readable instructions implemented on a non-transitory computer-readable memory comprised in the PMC. Such functional modules are referred to hereinafter as comprised in the PMC.

It is to be noted that the term processor referred to herein should be expansively construed to cover any processing circuitry with data processing capabilities, and the present disclosure is not limited to the type or platform thereof, or number of processing cores comprised therein. It should be noted that the system 100 can be connected to the measurement device in a wired or wireless connection.

According to certain embodiments, functional modules comprised in the PMC 102 can comprise a data analysis module 104 and a respiratory condition determining module 106 which are operatively connected there between. According to certain embodiments of the present disclosure, upon obtaining, from the device 130, input data indicative of an alternating signal representative of change of length of at least part of a contour along the subject's chest wall and/or abdominal wall during one or more breathing cycles of the subject, the data analysis module 104 can be configured to analyze the input data to detect an irregular breathing pattern. The irregular breathing pattern is represented by at least one local change in the form of the alternating signal during an expiratory phase of at least one of the one or more breathing cycles. The respiratory condition determining module 106 can be configured to determine a respiratory condition of the subject based on results of the detection.

In some embodiments, the system 100 can comprise a storage unit 122 configured to store data necessary for operating the system. The storage unit 122 can be configured to store the inputs to system 100, including the input data obtained from device 130, and/or predefined operation parameters. In some cases, the input can be pre-acquired from the device 130, and stored in the storage unit to be retrieved and processed by the PMC. The storage unit 122 can also be configured to store any of the intermediate and/or output processing results, such as, e.g., the analyzed data, detection result, determined respiratory conditions, etc. Alternatively, the storage unit 122 can reside external to system 100, e.g., in one of the external data repositories, or in an external system or provider, and the data can be retrieved via the I/O interface 126.

The I/O interface 126 can be configured to obtain the input data, and provide, as output, the detection result and/or the determined respiratory conditions. Optionally, system 100 can further comprise a graphical user interface (GUI) 124 configured to render display of the input and/or the output to the user. Optionally, the GUI can be configured to enable user-specified inputs for operating system 100.

In some embodiments, system 100 can be operatively connected to an external device, and the output of the system can be transmitted to the external device through wired or wireless communication. By way of example, the external device may be a monitoring device of a health provider (e.g. a doctor) that can use the transmitted information for intervention, e.g. by calling the patient and asking him/her to take certain medications. By way of another example, additionally or alternatively, the output may also be transmitted to an external device of the subject patient, such as, e.g., a mobile phone, a Tablet (e.g. iPad) or any other computation device thereof, so that the patient can take corresponding measures, such as, e.g. by taking medication or reducing the amount of smoking, etc. In some cases, the output information can also be transmitted to the cloud for storage or additional computation, such as, e.g., for training Machine Learning networks usable for various applications.

In some embodiments, for monitoring the patient and assessing the medical condition thereof, the system 100 can further include other sensors, such as, e.g. Inertial Measurement Unit (IMU), temperature sensor, etc., to provide additional health data on the medical condition of the subject. The system 100 can be used to monitor the patient at various times: e.g. during sleep, during regular activities, during sport, etc. It can be used at various settings including at home, at work, in hospital, in outpatient clinics, in elderly homes, nursing homes, etc.

It is also noted that the system illustrated in FIG. 1 can be implemented in a distributed computing environment. By way of example, some of the functional modules shown in FIG. 1 can be distributed over several local and/or remote devices, and can be linked through a communication network. By way of another example, system 100 can be located at a different location from the device 130. It is to be noted that although device 130 is exemplified in FIG. 1 as being external to system 100, in some cases, the functionality of the device 130, or at least part thereof, can be integrated with system 100. For instance, in some embodiments, system 100 can further comprise the device 130 and can be configured to perform the signal transmission and processing so as to generate the input data. In some other embodiments, the processing module of the device 130 can be integrated with the PMC of system 100, and system 100 can receive the signals from the receiver of device 130, and process the signals to obtain relevant measurements and generate the input data as required by system 100, as will be described below with reference to FIG. 2.

Those versed in the art will readily appreciate that the teachings of the presently disclosed subject matter are not bound by the systems illustrated in FIG. 1; equivalent and/or modified functionality can be consolidated or divided in another manner and can be implemented in any appropriate combination of software with firmware and hardware. The systems in FIG. 1 can be standalone network entities, or integrated, fully or partly, with other network entities. Those skilled in the art will also readily appreciate that the data repositories or storage unit therein can be shared with other systems or be provided by other systems, including third party equipment.

While not necessarily so, the process of operation of system 100 can correspond to some or all of the stages of the methods described with respect to FIGS. 2-4. Likewise, the methods described with respect to FIGS. 2-4 and their possible implementations can be implemented by system 100. It is therefore noted that embodiments discussed in relation to the methods described with respect to FIGS. 2-4 can also be implemented, mutatis mutandis as various embodiments of the system 100, and vice versa.

Turning now to FIG. 2, there is illustrated schematically a generalized flowchart of monitoring a respiratory condition in accordance with certain embodiments of the presently disclosed subject matter.

Certain input data can be obtained (202) (e.g., by the PMC 102 via I/O interface 126 illustrated in FIG. 1). The input data can be indicative of an alternating signal representative of change of length of at least part of a contour along a subject's chest wall and/or abdominal wall during one or more breathing cycles. The input data can be generated by periodically measuring the change of length during the breathing cycles.

The term contour used herein refers to an outline representing or bounding the shape or form of a curving figure. Specifically, in the present disclosure, the contour refers to the outline along the surface of the chest wall and/or abdominal wall. In some cases, the contour can refer to an outline encircling the entire chest wall and/or abdominal wall, such as, e.g., the chest circumference, while in some other cases, the contour can refer to an outline covering part of the surface of the chest wall and/or abdominal wall, e.g., the outline can be in the form of a curved line, or a snake-shaped line, or a meander line, or a curly line or an arc or a winding line, or a serpentine line. The term breathing cycle refers to the period for a subject to complete an inhalation (e.g., from the time the subject starts to inhale till the time that he/she has breathed in the maximum amount of air in the current breathing cycle) and an exhalation (e.g., from the time the subject starts to exhale till the time that he/she has breathed out the maximum amount of air in the current breathing cycle).

As aforementioned, in some embodiments, the input data can be generated by a measurement device, such as, e.g., the device 130 in FIG. 1. The change of length can be measured by the measurement device using an electrical signal. The electrical signal is characterized in that at least part of signal path thereof contours at least part of the chest wall and/or abdominal wall. It is to be noted that the change of length is measured along a line (not necessarily straight) on the surface of the chest wall and/or abdominal wall.

According to certain embodiments, the receiver in the measurement device can be configured to determine measurements of an attribute of the electrical signal received by the receiver at two time points, e.g., at the beginning and the end of a time interval during a breathing cycle. For instance, the time interval can be a fraction of the breathing cycle. By way of example, the time interval can be the period it takes for an individual to complete an inhalation. By way of another example, the inhalation period can be divided to a few sub-periods and the time interval can be a sub-period of the inhalation period. For example, if the full inhalation period takes, e.g., one second to complete on average, the time interval can be set to be 0.1 second or 0.01 second. By way of another example, the time interval can be the period it takes for an individual to complete an exhalation, or a sub-period of the exhalation period.

For instance, the belt or strap illustrated in FIG. 6A measures the chest circumference multiple times during a breathing cycle. The chest circumference may be measured periodically, e.g., every T milliseconds. For example, it can be set T=50 milliseconds. A typical breathing cycle can last e.g. 6-10 seconds, thus a chest strap may measure the chest circumference e.g. 120-200 times per breathing cycle. It is to be noted that the selection of the time interval can be determined based on the accuracy required for the measurement.

In some embodiments, the receiver is further configured to determine measurements of an attribute of a received signal, for a plurality of time intervals during one or more breathing cycles, e.g. where the determination can be performed periodically or repeatedly.

A change in length of the signal path during the time interval can be calculated based on the measurements of the attribute received at the beginning and the end of the time interval. As noted above, at least part of the signal path contours at least part of the chest wall and/or abdominal wall. Any change in the signal path length during the breathing cycle can be assumed to be due to a change in the length of the at least part of the signal path that contours the at least part of the chest wall and/or abdominal wall, because even if the signal path includes a part that does not contour the chest wall, the length of that part should not be affected by the breathing. Therefore, the calculation of the change in length of the signal path based on the measurements should be equivalent to a calculation of a change in path length between a transmitter and receiver attached on either end of the at least part of chest wall and/or abdominal wall contoured by the at least part of the signal path. In this case, the calculation of the change in distance can be based on measurements of an attribute of a signal received by the receiver attached on the end of the at least part of chest wall and/or abdominal wall contoured by the at least part of the signal path, at the beginning and the end of a time interval.

By way of example, the at least part of the signal path that contours at least part of the chest wall is on or beside at least part of a circumference of the chest wall and/or the abdominal wall, so that this at least part of the signal path can be said to be along (e.g. on or beside) at least part of the circumference of the chest wall and/or the abdominal wall. The term “circumference” (e.g. of the chest wall) is used to refer to the path around (e.g. the chest wall), and the term “length of circumference” or “circumference length” is used to refer to the length of the path. Therefore, using this assumption, the calculation of the change in length of the signal path based on the measurements should be equivalent to a calculation of a change in distance between a transmitter and receiver positioned on the circumference of the chest wall, on either end of the at least part of the circumference along which the at least part the signal path is assumed to be. In this case, the calculation of the change in distance can be based on measurements of an attribute of the signal received by the receiver positioned on the circumference, at the beginning and the end of a time interval. The signal can travel, for instance, through a strap between the transmitter and the receiver located at the two ends of the strap, as exemplified in FIG. 6A, or the signal can travel, for instance, wirelessly (e.g. via an air channel) between the transmitter and the receiver which are located at the two ends of the air channel, as exemplified in FIGS. 5A and 5B.

According to certain embodiments, the attribute utilized for the measurement can be selected from a group comprising: the phase of the signal, time arrival of the signal, the amplitude of the signal, decay time of the signal, the degree of attenuation of the signal, time duration between transmission and reception of the signal, or any other difference between transmission and reception. Examples of various attributes and the associated calculation thereof can be found in U.S. Pat. No. 10,194,835 titled “METHOD OF MONITORING VOLUMETRIC CHANGE OF A LUNG”, which is hereby incorporated by reference herein by its entirety. It is to be noted that the subject matter does not limit the calculation of the change in the signal path during the time interval based on the measurements, and therefore the calculation can vary depending on the attribute, and/or for any other reason.

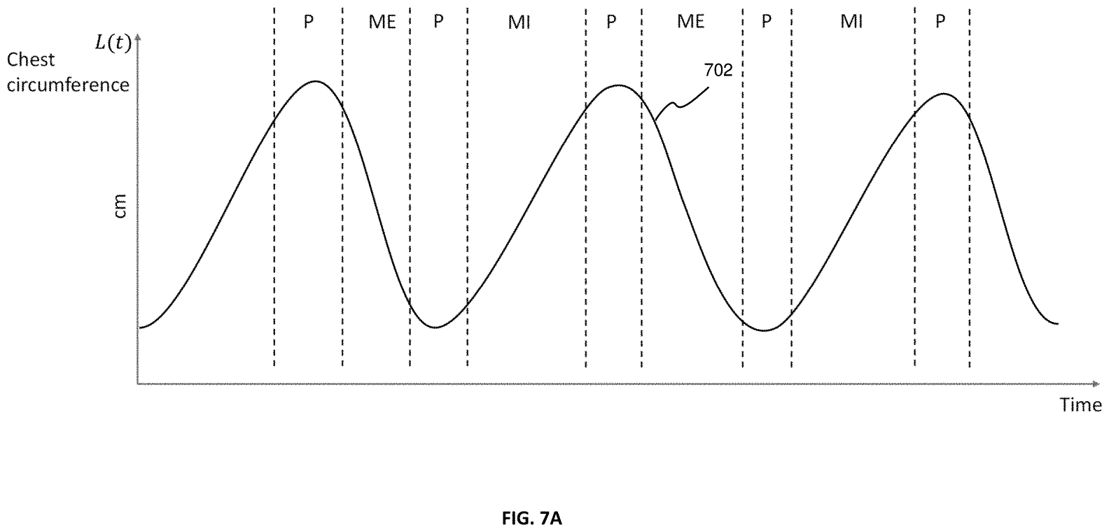

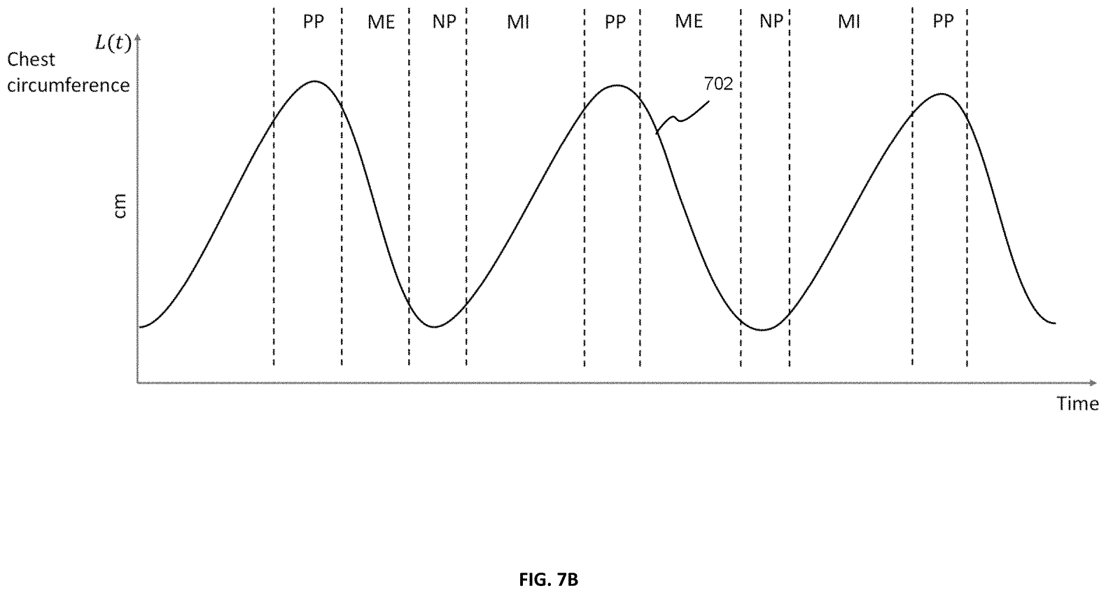

Referring now to FIGS. 7A and 7B, there are illustrated examples of input data in the form of an alternating signal in accordance with certain embodiments of the presently disclosed subject matter.

As illustrated in FIG. 7A, an alternating signal 702 denoted as L(t) represents change of length of at least part of a contour along a subject's chest wall during one or more breathing cycles. Specifically, in the present example, L(t) represents a change of chest circumference of the subject during the breathing cycles. In general, when the subject inhales, the chest expands and the chest circumference increases. When the subject exhales, the chest contracts and the chest circumference decreases. A measurement device such as a belt or strap illustrated in FIG. 6A can measure the chest circumference multiple times during a breathing cycle. For example, the chest circumference may be measured periodically every T milliseconds. The chest circumference can be measured in centimeters or any other length unit. The time period in which a subject exhales is referred to as an expiratory phase or stage, and the time period in which the subject inhales is referred to as inspiratory phase or stage.

In some cases, the time axis and the alternating signal can be divided into sections. The sections may represent different stages in the breathing cycle. It may also represent different stages within the breathing cycles. For example, as illustrated in FIG. 7A, the stages may include, e.g., “Peak”, denoted in the figure as P, “Mid Expiratory stage”, denoted in the figure as ME, and “Mid Inspiratory stage”, denoted in the figure as MI. The peak may refer to a positive peak or a negative peak, depending on the amplitude value of the peak. In the example as illustrated in FIG. 7B, the stages include “Positive Peak”, denoted in the figure as PP, “Mid Expiratory stage”, denoted in the figure as ME, “Negative Peak”, denoted in the figure as NP, and “Mid Inspiratory stage”, denoted in the figure as MI.

The border point between two adjacent sections in the signal can be determined, for example, by looking for a point in which the absolute value of the slope of L(t), or absolute value of the derivative

$$\frac{\partial L ( t )}{\partial t}$$

of L(t) with respect to time, crosses a certain threshold. By way of another example, the border point between two adjacent sections, e.g., between PP and ME, in the signal can be determined by looking for a point in which the circumference crosses a value which represents e.g., the value of the negative peak plus 90% of the difference between the positive peak and the negative peak.

In some cases, there could also be a gap between two adjacent sections in the circumference signal. For example, the period between t=10.0 sec to t=11.0 sec can be determined as “Peak”, and the period between 11.3 sec and 14.2 sec can be determined as “Mid Inspiratory stage”, but the period between 11.0 to 11.3 will not be part of any stage.

Continuing with the description of FIG. 2, upon obtaining the input data, the input data can be analyzed (204) (e.g., by the data analysis module 104 illustrated in FIG. 1) to detect an irregular breathing pattern. The irregular breathing pattern can be represented by at least one local change in the form of the alternating signal during an expiratory phase of at least one of the one or more breathing cycles. A respiratory condition of the subject can be determined (206) (e.g., by the respiratory condition determination module 106 illustrated in FIG. 1) based on a result of the detection.

According to certain embodiments of the presently disclosed subject matter, in order to determine the respiratory condition of a patient, it is possible to continuously monitor the alternating signal during the breathing cycles, and detect irregular breathing patterns thereof during specific stages of the breathing cycles. Such irregular patterns can be an indication of certain lung diseases, such as, e.g., asthma, or chronic obstructive pulmonary disease (COPD), or Congestive Heart Failure (CHF), or infectious diseases such as Covid-19 and Pneumonia etc. Such irregular patterns can also be an indication of certain episodes of deterioration, or exacerbation, or inflammation, or improvements, or response to treatment, of the respiratory condition of a patient suffering from such diseases. In such diseases, early detection of exacerbations can prevent deterioration and unnecessary hospitalization.

By way of example, when a patient is having an asthma attack, which is also called an asthma exacerbation, the airways become swollen and inflamed. The muscles around the airways contract and the airways produce extra mucus, causing the breathing tubes to narrow. During the attack, the patient may cough, wheeze or have trouble breathing air out from the lungs. Based on clinical tests and experiments, the present disclosure has revealed that during an asthma attack, there is presence of one or more local changes in the alternating signal during an expiratory phase of one or more breathing cycles. By way of example, the at least one local change can comprise one or more of the following: local bend, local peak, and change of slope.

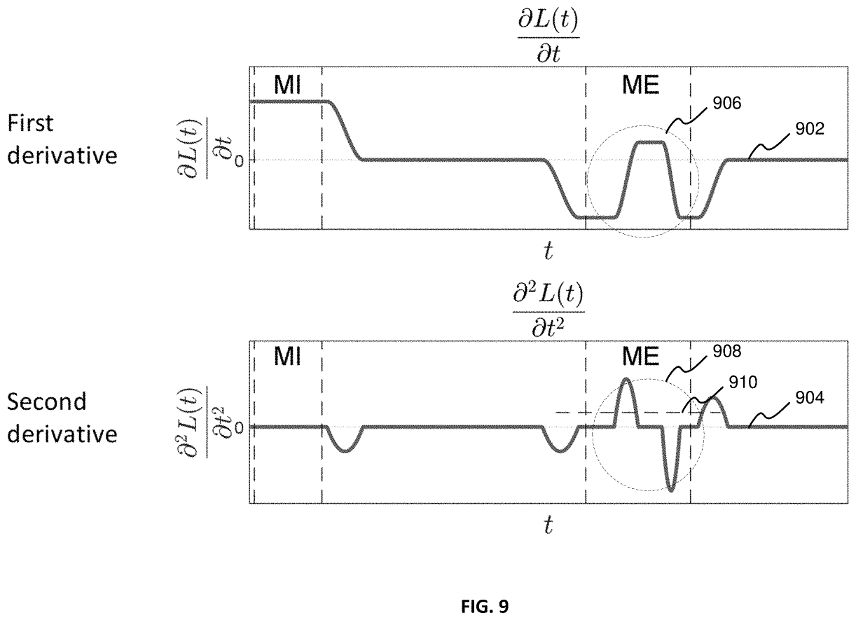

According to certain embodiments, the at least one local change can be detected by calculating first derivative data indicative of change rate of the length of at least part of a contour with respect to time. In some cases, the at least one local change can be detected by calculating second derivative data indicative of a change rate of the first derivative data with respect to time. In some further cases, the at least one local change can be detected by a combination of both the first and the second derivative data. In some embodiments, it can be regarded that the at least one local change is characterized by the second derivative data thereof passing a predefined threshold, as will be exemplified with reference to FIGS. 9, 12 and 14. In some embodiments, it can be regarded that the at least one local change is characterized by the absolute value of the second derivative data thereof passing a predefined threshold.

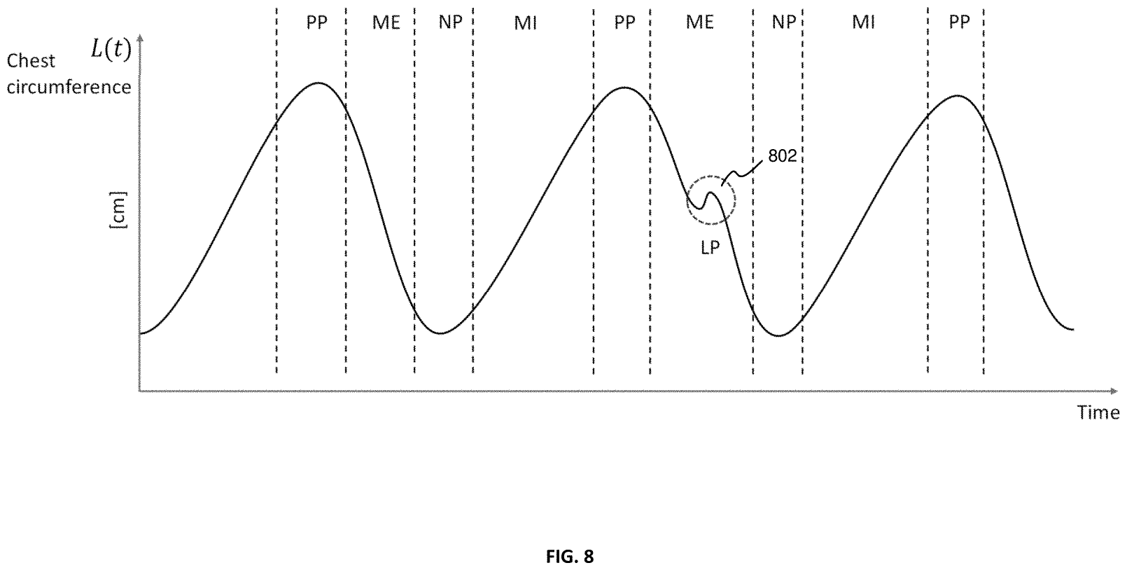

Referring now to FIG. 8, there is illustrated an example of a local peak in the form of the alternating signal during an expiratory phase of a breathing cycle in accordance with certain embodiments of the presently disclosed subject matter.

FIG. 8 depicts an example of an irregular breathing pattern represented by a local peak. As shown, during the second ME period of the alternating signal, there is the presence of two local peaks 802 marked with a dashed circle and denoted as LP, including a positive local peak and a negative local peak within the ME period. A positive local peak refers to a summit point right before which the signal is increasing or non-decreasing in value, and right after which the signal is decreasing or non-increasing in value within the ME period. A negative local peak refers to a valley point right before which the signal is decreasing or non-increasing in value, and right after which the signal is increasing or non-decreasing in value within the ME period. The existence of such a local peak can be regarded as an irregular breathing pattern or event, which can be used to indicate a pathological respiratory condition of a lung disease patient, such as, e.g., asthma.

Since local peaks, and Positive and Negative Peaks (PP and NP) are all reflected as peaks in the alternating signal, in order to detect the local peaks, it is needed to distinguish a positive local peak from PP, and distinguish a negative local peak from NP.

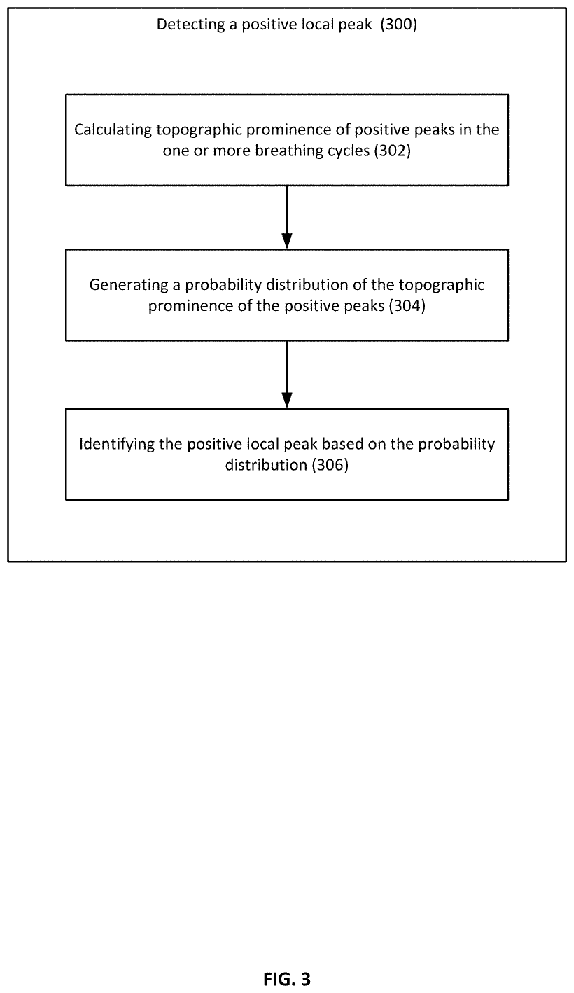

Referring now to FIG. 3, there is illustrated a generalized flowchart of detecting a positive local peak in accordance with certain embodiments of the presently disclosed subject matter.

According to certain embodiments, the positive local peak can be detected (300) (e.g., by the data analysis module 104) by calculating (302) topographic prominence of positive peaks in the one or more breathing cycles, generating (304) a probability distribution of the topographic prominence of the positive peaks, and identifying (306) the positive local peak, or determining a respiratory condition based on the probability distribution. By way of example, the probability distribution can be in the form of a probability mass function, or a probability density function of the prominence values. For instance, the positive peaks with low topographic prominence (e.g., relative to the average topographic prominence of all the positive peaks) can be marked as positive local peaks.

In some embodiments, the positive local peak can be detected by searching for two consecutive positive peaks in the alternating signal using the first derivative data, where a time difference between the two consecutive positive peaks is less than a predetermined threshold. For instance, the time difference between these two positive peaks is below αT, where T is the average time of a breathing cycle, and a (0<α<1) is a constant. For example α=0.2.

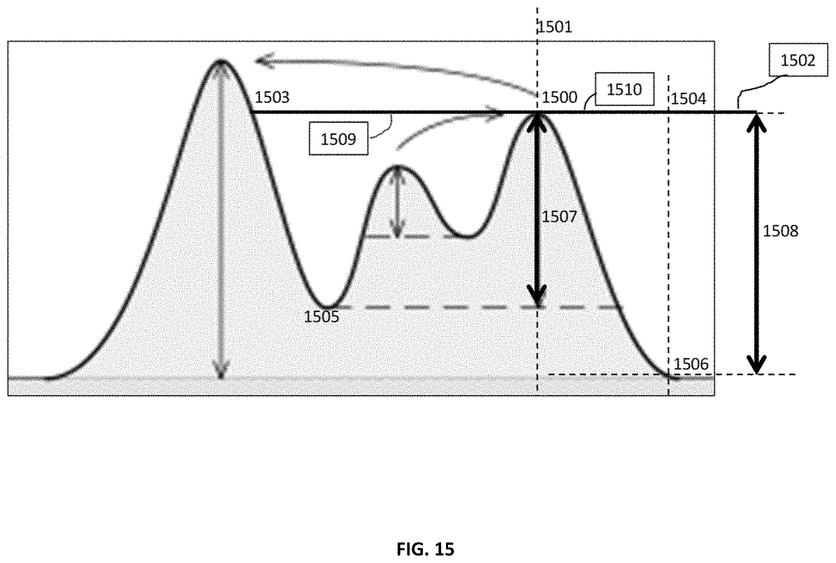

Referring now to FIG. 15, there is illustrated an example of measuring the prominence of a peak in accordance with certain embodiments of the presently disclosed subject matter.

The prominence, or topographic prominence, of a peak refers to the minimum vertical distance that the signal must descend on either side of the peak before either climbing back to a level higher than the peak, or reaching an endpoint. The prominence of a peak measures how much the peak stands out due to its intrinsic height and its location relative to other peaks. A low isolated peak can be more prominent than one that is higher, but is an otherwise unremarkable member of a tall range.

There are various methods of measuring the prominence of a peak, and there is now described an example of measurement:
Step 1: Place a vertical marker line 1501 on the peak 1500.
Step 2: Extend a horizontal line 1502 from the peak 1500 to the left and right until the line does one of the following:
   Crosses the signal because there is a higher peak 1503;
   Reaches the left or right end of the signal 1504;
Step 3: Find the minimum of the signal in each of the two intervals 1509 and 1510: the minimum 1505 in the interval 1509 and the minimum 1506 in the interval 1510). This point is either a valley or one of the signal endpoints.
Step 4: The higher of the two minimums 1505 specifies the reference level. The height 1507 of the peak above this level is its prominence.

Alternatively, Step 4 as described above can be implemented differently as below:
First alternative of Step 4: The minimum of the interval to the right 1506 specifies the reference level. The height of the peak above this level 1508 is its prominence (or termed as prominence relative to the right).
Second alternative of Step 4: The minimum of the interval to the left 1505 specifies the reference level. The height of the peak above this level 1507 is its prominence (or termed as prominence relative to the left).

In some embodiments, the at least one local change further comprises a negative local peak, such as in the example illustrated in FIG. 8. In such cases, in order to detect the negative local peak, the alternating signal can be modified by multiplying the signal with a negative number so as to transform the negative peaks into positive peaks, and the calculating, generating and identifying as described above with reference to FIG. 3 can be performed on the modified alternating signal to detect the negative local peaks. Similarly, the negative local peaks can also be detected by searching for two consecutive negative peaks in the alternating signal using the first derivative data, where a time difference between the two consecutive negative peaks is less than a predetermined threshold.

In some further embodiments, the local peaks can be detected by using first derivative data and/or second derivative data, as described above. Referring to FIG. 9, there is illustrated schematically detection of local peaks using first derivative data and/or second derivative data in accordance with certain embodiments of the presently disclosed subject matter.

As shown, the first derivative data 902 is generated based on the corresponding alternating signal data. As described above, the first derivative data can be calculated as the change rate of the length of at least part of the contour along the chest wall and/or abdominal wall with respect to time. The second derivative data 904 is generated based on the first derivative data 902. For instance, the second derivative data can be calculated as a change rate of the first derivative data with respect to time. In the presence of a negative local peak and a positive local peak during the expiratory stage, as exemplified in FIG. 8, the corresponding signal pattern in the first derivative data, as marked by a dashed circle and denoted as 906, can be used to detect the local peaks. As seen, the first derivative signal crosses zero twice within the ME stage, the first crossing representative of the negative local peak, and the second crossing representative of the positive local peak. Alternatively or additionally, the corresponding signal pattern in the second derivative data, as marked by a dashed circle and denoted as 908, can be used to detect the local peaks. As shown, the second derivative signal goes above a certain positive threshold 910 and then goes below a certain negative threshold during the expiratory phase.

According to certain embodiments, the number of occurrences of the local peak during the one or more breathing cycles can be counted, and the respiratory condition can be determined based on the counted number. For example, when a patient is in a respiratory deterioration condition under asthma attack, it could be that during a period of N minute (e.g. N=3), there will be M1 breathing cycles which contain a local peak within the ME stages. When the patient's condition improves, during the same-length period of N minutes, there will only be M0 breathing cycles which contain local peaks within the ME stages, where M0<M1.

According to certain embodiments, the number of occurrences of the local peak during the one or more breathing cycles can be counted, such that any sequence of two or more LPs where the time difference between each two neighboring LPs is below βT where T is the average time of a breathing cycle, and β (0<β<1) is a constant, is counted as only one occurrence. For example, β=0.45. The respiratory condition can then be determined based on the counted number.

It is to be noted that the above data analysis methods for detecting local peaks are for exemplary purposes only and should not be regarded as limiting the present disclosure in any way. Other appropriate methods capable of detecting local peaks can be used in addition to or in lieu of the above.

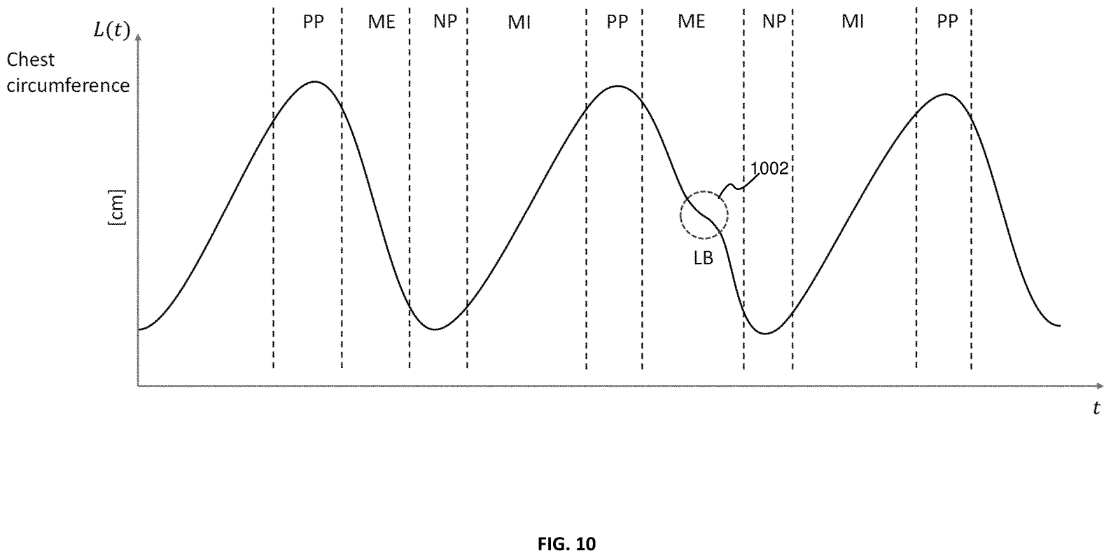

According to certain embodiments, the at least one local change can comprise a local bend. Referring now to FIG. 10, there is illustrated an example of local bend in the form of the alternating signal during an expiratory phase of a breathing cycle in accordance with certain embodiments of the presently disclosed subject matter.

FIG. 10 depicts an example of an irregular breathing pattern represented by a local bend. As shown, during the second ME period of the alternating signal, there is presence of a local bend 1002 marked with a dashed circle and denoted as LB. A local bend refers to the signal having two curves extending to different directions without a summit point between them. The existence of a local bend can be regarded as an irregular breathing pattern or event, which can be used to indicate a pathological respiratory condition of a lung disease patient.

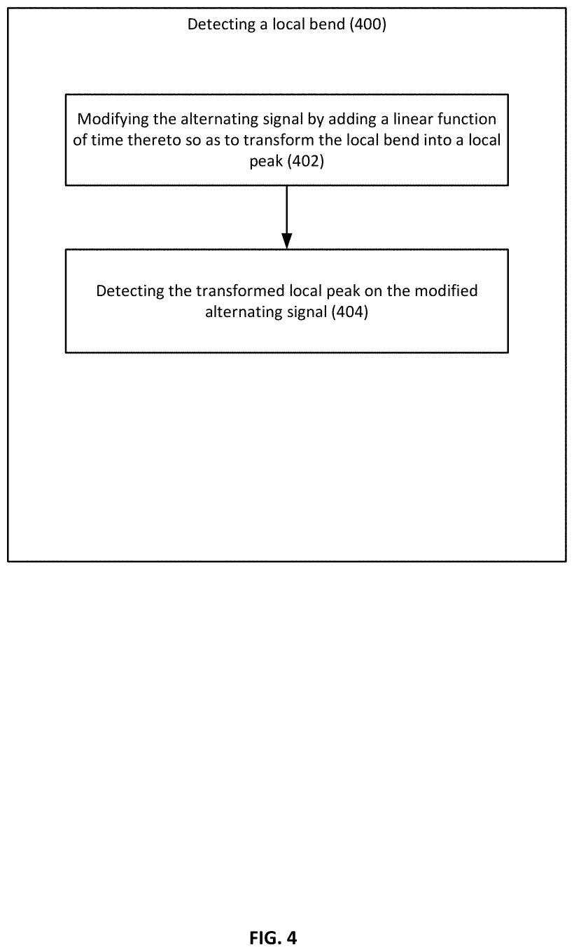

Referring now to FIG. 4, there is illustrated a generalized flowchart of detecting a local bend in accordance with certain embodiments of the presently disclosed subject matter. According to certain embodiments, the local bend can be detected (400) (e.g., by the data analysis module 104) by modifying (402) the alternating signal by adding a linear function of time to the alternating signal so as to transform the local bend into a local peak, and detecting (404) the transformed local peak on the modified alternating signal.

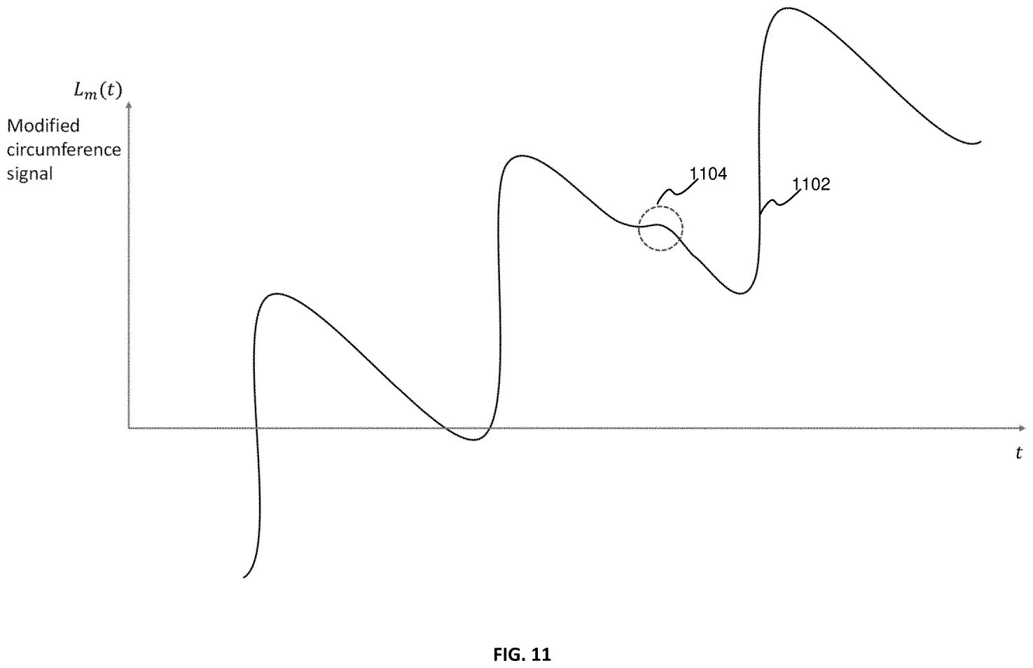

Referring now to FIG. 11, there is illustrated an example of a modified alternating signal in accordance with certain embodiments of the presently disclosed subject matter.

By way of example, a modified-circumference signal Lₘ(t) 1102 can be generated from the alternating signal L(t) by adding a linear function of time as follows: Lₘ(t)=L(t)+k×t, where k is constant (k can be positive or negative). It can be seen that by proper choice of k, the local bend 1002 in L(t) becomes a positive local peak 1104 in Lₘ(t). Once the local bend becomes a local peak, it is possible to use the method of detecting a local peak as described with reference to FIG. 3 to detect the local peak, e.g., by calculating the topographical prominence of the peaks, and using the prominence values as a measure for irregularity of the breathing cycle.

Additionally or alternatively, a modified alternating signal Lₘ(t) can be generated from the alternating signal L(t) by multiplying L(t) by a negative number, such as, e.g., −1, and adding a linear function of time as follows: Lₘ(t)=−L(t)+k×t, where k is constant (k can be positive or negative).

It should be noted that with a modified alternating signal Lₘ(t)=L(t)+k×t with a positive value of k, as k increases, more local bends in the ME stage become local peaks. A larger value of k means that even subtle bends can become peaks. This means that by proper choice of that value, it is possible to control the level of sensitivity of the algorithm to subtle bends, or subtle irregularities, or subtle shaking of the alternating signal during the expiratory phase.

It should also be noted that in some embodiments, additional or alternative detection of local changes can be performed in the MI stage. With a modified alternating signal Lₘ(t)=L(t)+k×t with a negative value of k, as the absolute value of k increases, (that is, k decreases,) more local bends in the MI stage will become local peaks. Similar detection can be performed in the MI stage to detect the local bends.

According to certain embodiments, the at least one local change can comprise one or more local bends, and the one or more local bends can be detected by: iteratively modifying the alternating signal by adding respective linear functions of time, giving rise to respective modified alternating signals, detecting the one or more local bends based on a modified alternating signal selected from the respective modified alternating signals which maximizes the number of positive peaks in the one or more breathing cycles in the selected modified alternating signal.

Specifically, multiple values of the constant k corresponding to multiple linear functions can be used to generate multiple modified alternating signals having multiple “views” with different numbers of transformed peaks and their prominence. For example, the iterative process can include the following steps, with reference to the function of modified alternating signal: Lₘ(t)=L(t)+k×t:

Step 1: Set k=0, and count the number of positive peaks.

Step 2: Increase k by Δk and count the number of positive peaks again. Identify which of the peaks are new—relative to the peaks of k=0, and which of the peaks are old, in the sense that they already existed in k=0.

Step 3: If the number of peaks did not decrease relative to the previous value of k, repeat step 2.

Step 4: When reaching the highest value of k where the number of peaks is still non-decreasing relative to the previous value of k, count the number of occurrences of the local peak on the modified alternating signals in the one or more breathing cycles, and then determine the respiratory condition based on the counted number. For example, when a patient is in a respiratory deterioration condition under asthma attack, it could be that during a period of N minute (e.g. N=3), there will be M1 breathing cycles which contain a local peak in the modified alternating signal within the ME stages. When the patient's condition improves, during the same-length period of N minutes, there will only be M0 breathing cycles which contain local peaks within the ME stages, where M0<M1.

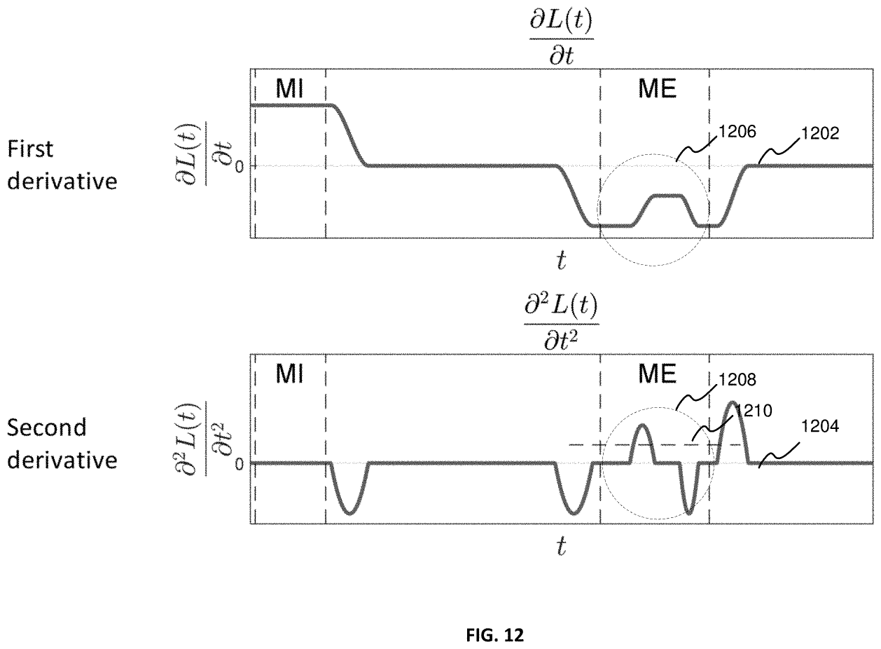

In some embodiments, the local bends can be detected by using first derivative data and/or second derivative data, as described above. Referring to FIG. 12, there is illustrated schematically detection of local bends using first derivative data and/or second derivative data in accordance with certain embodiments of the presently disclosed subject matter.

As shown, the first derivative data 1202 is generated based on the corresponding alternating signal data. The second derivative data 1204 is generated based on the first derivative data 1202. In the presence of a local bend during the expiratory stage, as exemplified in FIG. 10, the corresponding signal pattern in the first derivative data, as marked by a dashed circle and denoted as 1206, can be used to detect the local bend. As shown, the first derivative signal suddenly increases, then decreases again, but does not cross the value of zero during the ME stage. Alternatively or additionally, the corresponding signal pattern in the second derivative data, as marked by a dashed circle and denoted as 1208, can be used to detect the local bend. As shown, the second derivative signal goes above a certain positive threshold 1210 and then goes below a certain negative threshold during the expiratory phase.

According to certain embodiments, the number of occurrences of the local bend during the one or more breathing cycles can be counted, and the respiratory condition can be determined based on the counted number. For example, when a patient is in a respiratory deterioration condition under asthma attack, it could be that during a period of N minute (e.g. N=3), there will be M1 breathing cycles which contain at least one local bend within the ME stages. When the patient's condition improves, during the same-length period of N minutes, there will only be M0 breathing cycles which contain at least one local bend within the ME stages, where M0<M1.

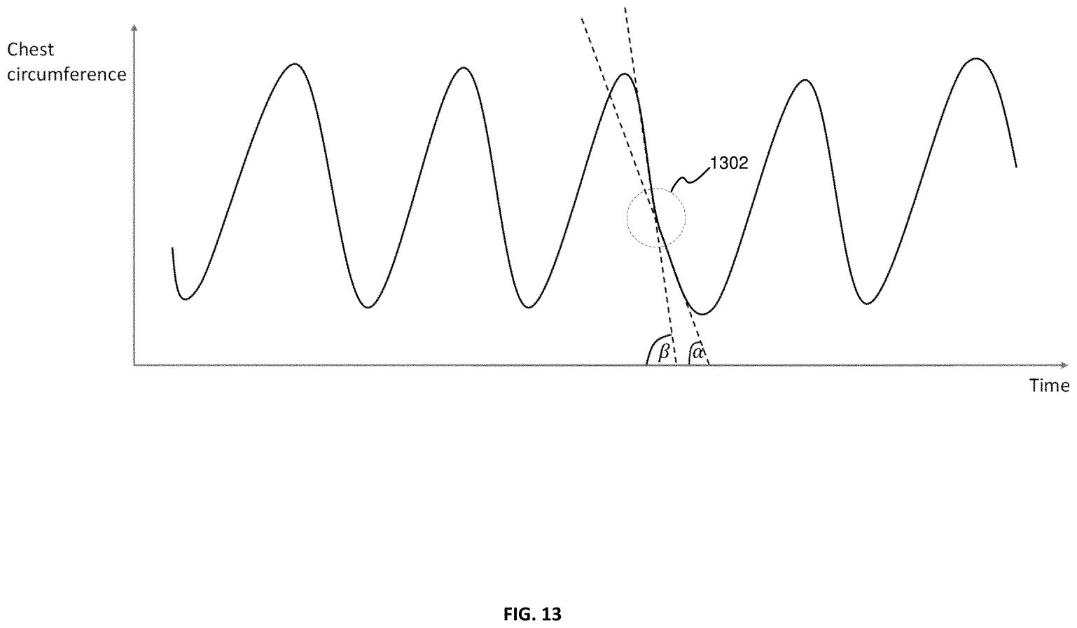

According to certain embodiments, the at least one local change can comprise a local change of slope. Referring now to FIG. 13, there is illustrated an example of a local change of slope in the form of the alternating signal during an expiratory phase of a breathing cycle in accordance with certain embodiments of the presently disclosed subject matter.

FIG. 13 depicts an example of an irregular breathing pattern represented by a local change of slope. As shown, during the third ME period of the alternating signal, there is presence of a local change of slope 1302 marked with a dashed circle. A local change of slope refers to the alternating signal changing slope at a certain point during the ME stage. As illustrated, at the beginning of the ME stage, the slope of the signal is −β, and it is relatively stable. The alternating signal remains in line with the dashed line for a long period of time. At a certain point, the slope of the signal changes to −α, and after this point, the slope again remains relatively constant for a long period of time, until reaching the NP stage. The existence of a local change of slope can be regarded as an irregular breathing pattern or event, which can be used to indicate a pathological respiratory condition of a lung disease patient.

It should be noted that the slope of the signal always changes at the vicinity of the peaks (PP and NP), therefore it is needed to distinguish the local change of slope within ME from changes in slope at the vicinity of the PP or NP.

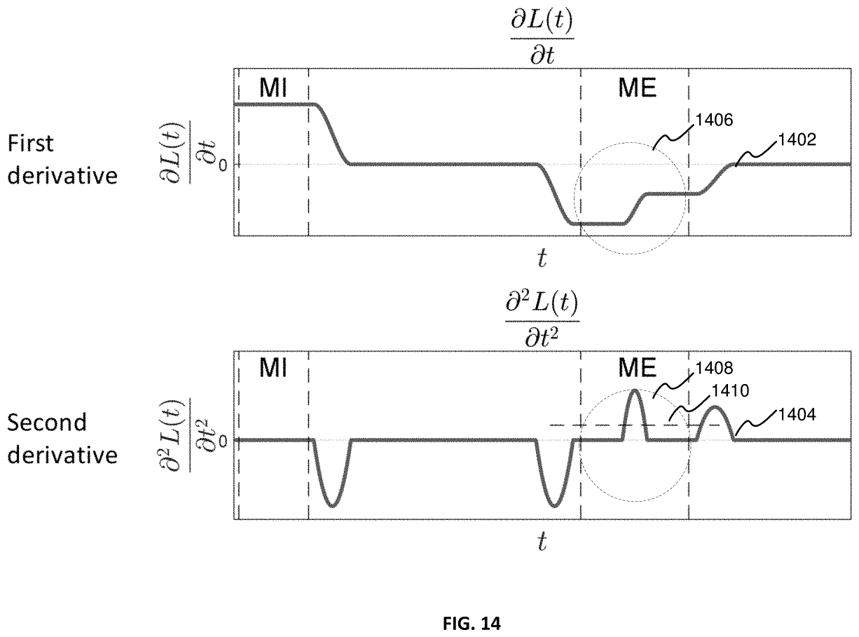

In some embodiments, the local change of slope can be detected by using first derivative data and/or second derivative data, as described above. Referring to FIG. 14, there is illustrated schematically detection of a local change of slope using first derivative data and/or second derivative data in accordance with certain embodiments of the presently disclosed subject matter.

As shown, the first derivative data 1402 is generated based on the corresponding alternating signal data. The second derivative data 1404 is generated based on the first derivative data 1402. In the presence of a local change of slope during the expiratory stage, as exemplified in FIG. 13, the corresponding signal pattern in the first derivative data, as marked by a dashed circle and denoted as 1406, can be used to detect the local change of slope. As shown, the first derivative signal suddenly increases, then stays at the increased level, but does not cross zero. Alternatively or additionally, the corresponding signal pattern in the second derivative data, as marked by a dashed circle and denoted as 1408, can be used to detect the local change of slope. As shown, the second derivative signal goes above a certain positive threshold 1410 and then returns to zero.

According to certain embodiments, the local change of slope can be detected by searching for two events of change of slope where the time difference between these two changes is below αT, where T is the average time of a breathing cycle, and α (0<α<1) is a constant. For example, α=0.2.

It is to be noted that the above described attributes and/or methods used for detection of the local changes, such as the first derivative, second derivative, time difference, prominence, etc., are illustrated for exemplary purposes only, and should not be regarded as limiting the present disclosure in any way. Any other suitable attributes and/or detection methods can be used in addition to or in lieu of the above.

It is also to be noted that although the alternating signal is illustrated in certain figures as a chest circumference signal, this is only one example of a possible alternating signal. The present disclosure is also applicable to other signals representative of any other part of a contour along the chest wall and/or abdominal wall, such as, e.g., part of a contour starting from the surface of the chest wall and ending at the surface of the abdominal wall.

According to certain embodiments, the monitoring process as described with reference to FIG. 2 can further comprise counting the number of occurrences of the irregular breathing pattern during the one or more breathing cycles, and determining the respiratory condition based on the counted number. In some embodiments, the input data is analyzed to detect an additional irregular breathing pattern represented by irregular change of length of at least part of a contour between the beginning and end of the expiratory phase. In some embodiments, the method for detecting the patient's condition may optionally utilize the information regarding the patient's posture or position or activity during the breathing cycles, combined with the measurements of changes in the length of the contours. For example, the measurement of a local peak can be different when the patient is in a sitting position or a standing position, and the threshold can be set differently depending on the specific position of the patient.

In some cases, the respiratory condition can be selected from a list of pre-defined set of conditions indicative of different types of deterioration of lung function. In some cases, an alert can be generated upon determination of a pre-defined respiratory condition, and the alert can be sent to an external device, such as, e.g., a monitoring device at the health provider, and/or a mobile device of the patient, etc.

In some embodiments, the respiratory condition of the subject, and the clinical condition related thereto, can be determined in combination with one or more additional parameters and/or metrics. For purpose of illustration, there are listed below examples of metrics usable in this regard:
Breathing signal amplitude, (or amplitude after removal of the mean value of the amplitude).
Breathing signal Root Mean Square (RMS), or standard deviation (STD), or power, or variance.
Average length (time duration) of ME, average length of ME relative to average breathing cycle time, or average length of ME relative to average length of MI.
Average length (time duration) of MI, average length of ME relative to average breathing cycle time, or average length of MI relative to average length of ME.
Changes in the mean value (or more specifically, to monitor a low frequency component of the signal in the breathing cycle. For example, if the breathing cycle time is T, a low-pass filtered version of the signal can be used, e.g. by filtering the signal with a moving-average type of low-pass filter, where the moving average time window is 10T, or 1 minute). This metric can also indicate the amount of air which exists in the lungs. For example, during normal breathing, the air volume can change between 4000 cc at NP to 4500 cc at PP and have a mean value of 4250 cc, but during exacerbation, the air volume can change between 4200 cc at NP to 4700 cc at PP and have a mean value of 4450 cc, or 3800 cc at NP to 4300 at PP and have a mean value of 4050 cc. Although these numbers represent volume of the lung, they can be reflected on the change of length of the contour along the chest wall and/or abdominal wall. These mean values, or changes thereof, can be used to assist the determination of the respiratory condition.
Breathing signal period or breathing rate.

It should be noted that the change of length of the contour, as well as the above described metrics, which are used to identify the respiratory condition, can be either compared with a certain absolute threshold, or with a baseline condition, wherein the baseline condition refers to the normal condition of the patient. For example, for an asthma patient, the baseline condition can be obtained when the patient is not having an asthma attack.

It is to be noted that although certain embodiments of the present disclosure refer to detecting an irregular breathing pattern in the ME stage, in some cases, irregular patterns such as, e.g., local peaks, can be identified during other stages of the breathing cycle, such as MI, PP or NP, and used for indication of specific respiratory conditions. Therefore, the presently disclosed subject matter can be applicable to detection of such irregular patterns in these stages in a similar manner.

One of the technical advantages resulting from certain aspects of the present disclosure is the specific measurement data used as input for data analysis and determination of respiratory condition, e.g., input data indicative of an alternating signal representative of change of length of at least part of a contour along a subject's chest wall and/or abdominal wall, as can be measured by a measurement device 130 illustrated in the figures. This type of input data, as compared to other types of respiratory data such as, e.g., flow meter data, IMU sensor data, temperature data, impedance data, etc., has the advantage of being easily obtainable, having a high level of measurement accuracy, has been proven to have a close correlation with the volumetric change of the lung during the breathing cycles, and is thus advantageous for continuous monitoring of the respiratory condition.

Another technical advantage resulting from certain aspects of the present disclosure, is using the one or more local changes during the expiratory stage of the breathing cycles to represent specific irregular breathing patterns. These local changes, such as local peak, local bend and local change of slope, are proved by the present disclosure to be more closely correlated to the respiratory condition of the patient and the specific type of measurement data as described above, as compared to other conventional medical indications, such as, e.g., length of the expiratory stage and the inspiratory stage, etc.

A further technical advantage resulting from certain aspects of the present disclosure, is the specific data analysis methods used to detect the specific types of local changes. The data analysis steps, such as calculating specific attributes characterizing the signal, modifying/transforming the signal to other forms, etc., are performed on the input data for the purpose of transforming the input data into a different state and output, i.e., a meaningful and applicational output indicative of medical indication of the respiratory condition of a subject, thereby enabling further possible intervention and treatment based on the output.

It is appreciated that the examples and embodiments illustrated with reference to the respiratory monitoring system in the present description are by no means inclusive of all possible alternatives but are intended to illustrate non-limiting examples only.

It is to be understood that the present disclosure is not limited in its application to the details set forth in the description contained herein or illustrated in the drawings. The present disclosure is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the presently disclosed subject matter.

It will also be understood that the system according to the present disclosure may be, at least partly, implemented on a suitably programmed computer. Likewise, the present disclosure contemplates a computer program being readable by a computer for executing the method of the present disclosure. The present disclosure further contemplates a non-transitory computer readable memory or storage medium tangibly embodying a program of instructions executable by the computer for executing the method of the present disclosure.

The non-transitory computer readable storage medium causing a processor to carry out aspects of the present disclosure can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Those skilled in the art will readily appreciate that various modifications and changes can be applied to the embodiments of the present disclosure as hereinbefore described without departing from its scope, defined in and by the appended claims.

1. A computerized method of monitoring a respiratory condition of a subject, the method performed by a processor and memory circuitry (PMC) and comprising:
obtaining input data indicative of an alternating signal representative of change of length of at least part of a contour along a subject's chest wall and/or abdominal wall during one or more breathing cycles, the input data generated by periodically measuring the change of length during the breathing cycles;
analyzing the input data to detect an irregular breathing pattern, the irregular breathing pattern represented by at least one local change in the form of the alternating signal during an expiratory phase of at least one of the one or more breathing cycles; and
determining a respiratory condition of the subject based on a result of the detection.

2. The computerized method of claim 1, wherein the change of length is measured using an electrical signal, and at least part of signal path of the electrical signal contours at least part of the chest wall and/or abdominal wall.

3. The computerized method of claim 2, wherein the electrical signal flows through a band attachable to the chest wall and/or abdominal wall of the subject.

4. The computerized method of claim 2, wherein the input data is generated by, for at least one time interval during a breathing cycle, measuring an attribute of the electrical signal received at the beginning and the end of the time interval, and calculating a change in length of the signal path during the time interval based on the measurements of the attribute received at the beginning and the end of the time interval.

5. The computerized method of claim 1, wherein the at least one local change comprises one or more of the following: local bend, local peak, and change of slope.

6. (canceled)

7. The computerized method of claim 1, wherein the at least one local change is characterized by second derivative data thereof passing a predefined threshold, the second derivative data being indicative of change rate of first derivative data with respect to time, the first derivative data indicative of change rate of the length of at least part of a contour with respect to time.

8. The computerized method of claim 1, wherein the at least one local change comprises a positive local peak, and the analyzing comprises detecting the positive local peak by calculating topographic prominence of positive peaks in the one or more breathing cycles, generating a distribution of the topographic prominence of the positive peaks, and identifying the positive local peak based on the distribution.

9. The computerized method of claim 8, wherein the at least one local change comprises a negative local peak, wherein the analyzing further comprises detecting the negative local peak modifying the alternating signal by multiplying the alternating signal with a negative number so as to transform negative peaks in the one or more breathing cycles into positive peaks, and performing the calculating, generating and identifying on the modified alternating signal to detect the negative local peak.

10. The computerized method of claim 1, wherein the at least one local change comprises a positive local peak, and the analyzing comprises detecting the positive local peak by searching for two consecutive positive peaks using first derivative data, wherein a time difference between the two consecutive positive peaks is less than a predetermined threshold.

11. (canceled)

12. The computerized method of claim 1, wherein the at least one local change comprises one or more local bends, and the analyzing comprises iteratively modifying the alternating signal by adding respective linear functions of time, giving rise to respective modified alternating signals, detecting the one or more local bends based on a modified alternating signal selected from the respective modified alternating signals which maximize the number of positive peaks in the one or more breathing cycles in the selected modified alternating signal.

13. The computerized method of claim 1, wherein the at least one local change comprises a local change of slope, and the analyzing comprises detecting the local change of slope using first derivative data and/or second derivative data.

14. (canceled)

15. (canceled)

16. (canceled)

17. (canceled)

18. A computerized system of monitoring a respiratory condition of a subject, the system comprising a processor and memory circuitry (PMC) configured to:
obtain input data indicative of an alternating signal representative of change of length of at least part of a contour along a subject's chest wall and/or abdominal wall during one or more breathing cycles, the input data generated by periodically measuring the change of length during the breathing cycles;
analyze the input data to detect an irregular breathing pattern, the irregular breathing pattern represented by at least one local change in the form of the alternating signal during an expiratory phase of at least one of the one or more breathing cycles; and
determine a respiratory condition of the subject based on a result of the detection.

19. The computerized system of claim 18, wherein the change of length is measured using an electrical signal, and at least part of signal path of the electrical signal contours at least part of the chest wall and/or abdominal wall.

20. The computerized system of claim 19, further comprises a band attachable to the chest wall and/or abdominal wall of the subject which the electrical signal flows through.

21. (canceled)

22. (canceled)

23. (canceled)

24. (canceled)

25. The computerized system of claim 18, wherein the at least one local change comprises a positive local peak, and the PMC is configured to analyze the input data to detecting the positive local peak by calculating topographic prominence of positive peaks in the one or more breathing cycles, generating a distribution of the topographic prominence of the positive peaks, and identifying the positive local peak based on the distribution.

26. The computerized system of claim 25, wherein the at least one local change comprises a negative local peak, wherein the PMC is further configured to modify the alternating signal by multiplying the alternating signal with a negative number so as to transform negative peaks in the one or more breathing cycles into positive peaks, and perform the calculating, generating and identifying on the modified alternating signal to detect the negative local peak.

27. (canceled)

28. (canceled)

29. The computerized system of claim 18, wherein the at least one local change comprises one or more local bends, and the PMC is configured to analyze the input data to detect the one or more local bends by iteratively modifying the alternating signal by adding respective linear functions of time, giving rise to respective modified alternating signals, detecting the one or more local bends based on a modified alternating signal selected from the respective modified alternating signals which maximizes the number of positive peaks in the one or more breathing cycles in the selected modified alternating signal.

30. The computerized system of claim 18, wherein the at least one local change comprises a local change of slope, and the PMC is configured to analyze the input data to detect the local change of slope by using first derivative data and/or second derivative data.

31. (canceled)

32. (canceled)

33. (canceled)

34. (canceled)

35. A device capable of monitoring a respiratory condition of a subject, the device attachable to the subject's chest wall and/or abdominal wall and comprising a transmitter, a receiver, and a processor and memory circuitry (PMC) operatively connected thereto, wherein:
the transmitter is configured to transmit an electronic signal to the receiver during one or more breathing cycles;
the receiver is configured to receive the electronic signal transmitted from the transmitter; and
the PMC is configured to:
periodically measure change of length of at least part of a contour along the subject's chest wall and/or abdominal wall during the one or more breathing cycles based on the received electronic signal, and generate data indicative of an alternating signal representative of the change of length;
analyze the data to detect an irregular breathing pattern, the irregular breathing pattern represented by at least one local change in the form of the alternating signal during an expiratory phase of at least one of the one or more breathing cycles; and
determine a respiratory condition of the subject based on a result of the detection.

36. A non-transitory computer usable medium having a computer readable program code embodied therein, said computer readable program code adapted to be executed to perform the computerized method of claim 1.
